(19) United States
(12) Patent Application Publication
(10) Pub. No.: US 2021/0386979 A1
(43) Pub. Date: Dec. 16, 2021

(54) BALLOON CATHETER SYSTEM FOR INFUSION OF MICELLES AT HIGH PRESSURE

(71) Applicant: Caliber Therapeutics, LLC, New Hope, PA (US)

(72) Inventors: William R. BAUMBACH, New Hope, PA (US); Darren R. SHERMAN, New Hope, PA (US)

(73) Assignee: Caliber Therapeutics, LLC, New Hope, PA (US)

(21) Appl. No.: 17/282,714

(22) PCT Filed: Sep. 26, 2019
(86) PCT No.: PCT/US2019/053267

§ 371 Date: Apr. 2, 2021

Related U.S. Application Data
(60) Provisional application No. 62/741,421, filed on Oct. 4, 2018.

(51) Int. Cl. A61M 25/10

(52) U.S. Cl. CPC A61M 25/104; A61M 2025/1013; A61M 2025/105; A61M 25/10181

(57) ABSTRACT

A balloon catheter system for infusion of micelles at high pressure. The system includes a catheter with a drug eluting balloon with a perforated wall with numerous pores, a reservoir of nanoparticles in an aqueous solution disposed within the balloon or in fluid communication with the balloon. The particles may comprise drug loaded micelles, where the micelles are provided in the size range of 40 to 250 nm generally (0.040 μm to 0.250 μm), and the pores of the balloon wall are configured to allow passage of the micelles with a minimum of disruption, The pores are conical, with the diameter of the pore at the inside of the balloon wall smaller than the diameter of the pores at the outside of the balloon wall.

### FIELD OF THE INVENTIONS

The inventions described below relate to the field of treatment of vascular disease, and more specifically to the field of drug eluting balloons for the treatment of restenosis.

### BACKGROUND OF THE INVENTIONS

Our prior U.S. Pat. No. 8,696,644, entitled Balloon Catheter Systems For Delivery Of Dry Drug Delivery Vesicles To A Vessel In The Body, described a drug eluting balloon catheter system well-suited for the delivery of a suspension of nanoparticles, in particular rapamycin loaded micelles, to blood vessels of a patient to treat various vascular diseases.

### SUMMARY

The devices and methods described below provide for improved administration of a suspension of nanoparticles through the wall of a drug eluting balloon. The system includes a catheter with a drug eluting balloon with a perforated wall with numerous pores, and a reservoir of nanoparticles in an aqueous solution disposed within the balloon or in fluid communication with the balloon. The particles may comprise drug loaded micelles, where the micelles are provided in the size range of 40 to 250 nm generally (0.040 μm to 0.250 μm), and the pores of the balloon wall are configured to allow passage of the micelles with a minimum of disruption. The pores are conical, with the diameter of the pore at the inside of the balloon wall smaller than the diameter of the pores at the outside of the balloon wall. The devices and method may be used in conjunction with balloon angioplasty to treat a lesion in a blood vessel to prevent restenosis after angioplasty, in conjunction with stent placement to open a blood vessel obstructed by a lesion to prevent restenosis after stent placement, or it may be used to treat a lesion in a blood vessel without concurrent use of angioplasty or concurrent placement of a stent.

### BRIEF DESCRIPTION OF THE DRAWINGS

FIG. 1 illustrates the balloon catheter system.

FIG. 2 shows a cross section of the balloon, illustrating the conical shape of the pores in the balloon.

### DETAILED DESCRIPTION OF THE INVENTIONS

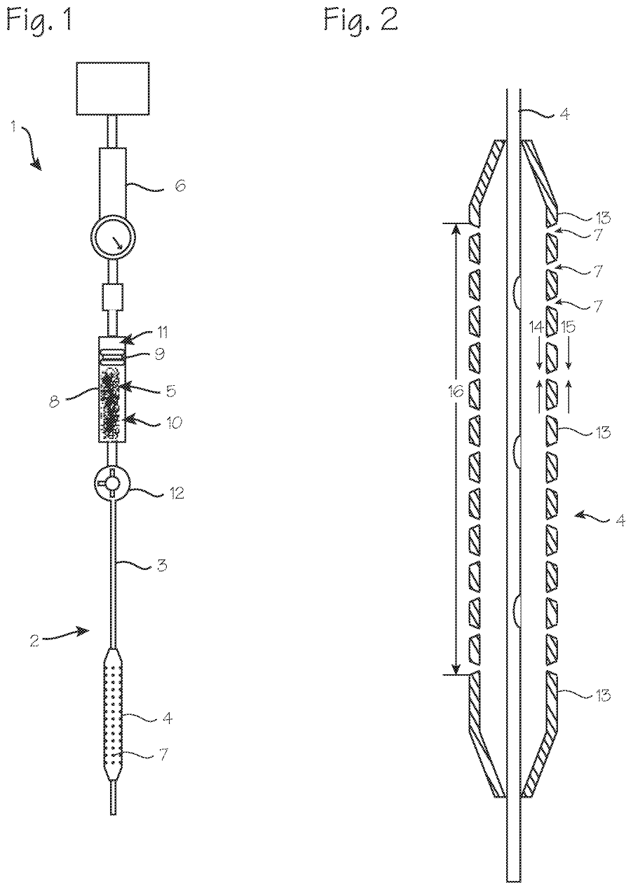

FIG. 1 illustrates the balloon catheter system 1, which includes a balloon catheter 2 comprising a catheter shaft 3 with a porous balloon 4 at the distal end of the catheter shaft, a reservoir 5 containing a suspension of nanoparticles, in particular micelles, loaded with a therapeutic agent, and an inflator 6 for forcing the suspension of micelles into the catheter and balloon, and through the walls of the balloon. The balloon wall is porous, with numerous pores 7 disposed over the surface of the balloon and passing through the balloon wall, communicating from the interior of the balloon to the exterior of the balloon. The balloon is suitable for introduction into the vasculature of a patient, to be placed in the coronary arteries, peripheral arteries, or elsewhere in the vasculature. The reservoir may be attached to the proximal end of the catheter shaft, in fluid communication with a lumen extending from the proximal end of the shaft to the interior of the balloon. The reservoir preferably comprise a syringe 8 with a piston 9 slidably disposed within the syringe to define a first chamber 10 which contains the suspension of nanoparticles, in fluid communication with a lumen balloon catheter shaft 4 and the interior space of the balloon, and a second chamber 11 which contains inflation fluid and is in fluid communication with the inflator 6. A stopcock or three-way valve 12 may be provided to vent the first chamber, or to connect to a stored vial or recently reconstituted suspension of nanoparticles, to fill the first chamber with the suspension of nanoparticles.

FIG. 2 shows a cross section of the balloon, illustrating the conical shape of the pores in the balloon. The balloon 4 has a wall 13 with numerous pores 7. The pores are generally conical in a longitudinal cross section (a conical cross section, along a long axis of the pores passing from an inside surface of the balloon wall to an outside surface of the balloon wall) shown in FIG. 2, and generally circular in transverse cross section. The pores have a first diameter 14 at the inside surface of the balloon wall, and a second diameter 15 at the outside surface of the balloon wall, and the first diameter is smaller than the second diameter. The first diameter, at the inside surface of the balloon, is preferably in the range of 3 to 8 μm (microns or p). The second diameter, at the outside surface of the balloon, is preferably in the range of 7 to 16 μm, (more preferably in the range of 7 to 12 μm) though larger than the first diameter. The wall thickness is preferable in the range of 15 to 28 μm, preferably about 21 μm thick. In an embodiment suitable for use in the coronary arteries of a patient, the cylindrical portion of the balloon may be 10 to 25 mm long, and expandable when pressurized to a diameter of about 2 to 5 mm. The number of pores may range from 100 to 400 pores for use in coronary arteries and 100 to 1000 pores for use in peripheral arteries, evenly distributed over the cylindrical portion of the balloon, optionally arranged in rows (5 to 15 rows) dispersed around the circumference of the balloon.

The micelles may be loaded with rapamycin or other therapeutic agents such as rapamycin analogs, ABT-578, zotarolimus, everolimus, biolimus A9, deforolimus (also referred to as ridaforolimus), temsirolimus, tacrolimus, pimcrolimus, nitric oxide synthase, C3 exoenzyme, RhoA inhibitors, tubulusin, A3 agonists, CB2 agonists, 17-AAG, Hsp90 antagonists, tyrphostins, cathepsin S inhibitors, paclitaxel, corticosteroids, glucocorticoids, dexamethasone, ceramides, dimethyl sphingosine, ether-linked diglycerides, ether-linked phosphatidic acids, sphinganines, estrogens, taxol, taxol analogs, actinomycin D, prostaglandins, vitamin A, probucol, Batimastat, Statins, Trapidil, mitomycin C and Cytochalasin B.

The nanoparticles used in this system and method described above should have a diameter in the range of 40 to 250 nm generally, and in the range of 60 to 120 nm when comprising micelles formulated from the tri-block copolymer mentioned above (PLGA-PEG-PLGA), as determined by dynamic light scattering techniques. This size will result in a balance of efficient penetration of the micelles into the artery walls and sufficient space within the micelles to encapsulate a suitable amount of rapamycin or other therapeutic substance. Use of tri-block polymers such as PLGA-PEG-PLGA will provide micelles in the desired size range. The ratio of micelle diameter to the first diameter is preferably in the range of 0.08 to 1 (approximately 1 to 12) to 0.005 to 1 (1 to 200), more preferably about 1 to 20. The systems and methods described above can be employed to deliver other small drug delivery vesicles or delivery vessels in addition to micelles, such as nanoparticles and liposomes.

Pressure applied by the inflator to the reservoir may be two to twenty atmospheres (203 kPa to 2027 kPa), and the inflator is preferably operated to apply 6 to 16 atmospheres (608 kPa to 1621 kPa) of pressure, more preferably 6 to 12 (608 kPa to 1216 kPa) atmospheres of pressure. With suspended micelle formulation in the suspension chamber, and holes sized and dimensioned as above, application of 12 atmospheres (1216 kPa) for 60 seconds will deliver the entire 1 ml of the suspended micelle formulation through the catheter and balloon wall. The pressure may be varied over the course of administration, for example, by applying pressure in the range of 6-8 atmospheres (608 kPa to 811 kPa) for about 20 seconds, and increasing pressure to 12 to 18 (1216 kPa to 1823 kPa) atmospheres for another 20 to 40 seconds (for an average of 12-14 atmospheres (1216 kPa to 1418 kPa) over the course of administration). The parameters may be adjusted to deliver 0.2 to 0.75 ml of suspension over the course of 10 to 120 seconds, preferably about 20 to 60 seconds, for flow rates of 0.0033 to 0.0375 ml/sec (preferred in the coronary arteries) or 0.0005 to 0.038 ml/sec (preferred in the peripheral arteries, out of the balloon for uptake by the surrounding blood vessel wall. The flow rate per pore is preferably in the range of range of 0.0001 to 0.00003 mL/sec/hole for coronary and 0.0001 to 0.00001 for peripheral arteries. These low flow rates help keep the balloon inflated so that it continues to exert opening force on the surrounding artery and maintains good contact with artery walls. Preferably, the total volume delivered is 0.2 ml to 0.75 ml. The dosage of drug or therapeutic agent actually delivered can thus be controlled and predetermined with some certainty by controlling the amount of drug or therapeutic agent in the micelle formulation disposed in the micelle storage chamber. For example, if it is desired to deliver 2 mg of rapamycin to a diseased portion of a blood vessel, the micelle reservoir containing 2 or 3 mg of rapamycin can be stored in the micelle storage chamber, reconstituting the micelles with fluid to achieve a concentration of 2 mg/ml (that is, 1 ml if the micelle storage chamber contains 2 mg total rapamycin), withdrawing 1 ml of fluid into the coiled tube suspension chamber, and forcing the entire 1 ml through the catheter and balloon into the blood vessel walls.

The ratio of the average particle size to the total pore area (on the at the inside surface of the balloon wall) may be controlled, to achieve a balance of internal balloon pressure needed to force compliance of the blood vessel to the balloon for angioplasty, flow rate of the suspension from the balloon to encourage uptake of the suspended micelles into surrounding vascular walls and avoid loss of the suspension in the blood stream. The total pore area may range from about 900 to about 30,000 microns (942 μm² (for example, 100 holes at 3 micron average diameter) to 25,120 μm² (1000 holes at 8 micron average diameter)). A very small ratio of average micelle particle diameter to inner wall total pore area in the range of 0.0000016 to 1 on the low side and 0.0008 to 1 on the large side will allow the suspension to be administered at high pressure, sufficient for angioplasty, while providing flow through the pores sufficient to treat the area with the loaded therapeutic agent. For example, with an average pore diameter of 5 microns (about 20.7 square microns) and a configuration of 200 total holes, the total pore area is 4142 μm² (4.142 million square nanometers), a particle size of 0.250 μm (250 nm), the ratio of particle size to total pore area on the inside wall would be 0.00006 to 1.

In use, the method of treating a diseased blood vessel includes inserting the balloon of the balloon catheter system into the blood vessel and forcing the suspension of nanoparticles into the balloon and through the pores to a blood vessel wall, using the inflator to apply pressure to the reservoir at high pressure, to force the suspension of nanoparticles into the balloon, and through the walls of the balloon. With pores configured as shown in FIGS. 1 and 2, and nanoparticles sized as described above, the nanoparticles will flow from the inside of the balloon to the outside of the balloon, while the balloon itself is inflated to sufficient pressure to apply force against the blood vessel walls sufficient to encourage uptake of the nanoparticles by the blood vessel wall and/or perform angioplasty. The method may be used in conjunction with balloon angioplasty to treat a lesion in a blood vessel to prevent restenosis after angioplasty, stent placement to open a blood vessel obstructed by a lesion to prevent restenosis after stent placement, or it may be used to treat a lesion in a blood vessel without concurrent use of angioplasty or concurrent placement of a stent.

To ensure that the entire length of a lesion is treated with the application of the therapeutic agent, the balloon used for the method is preferably longer than the lesion to be treated, such that the porous region of the balloon 16 (the region perforated with the numerous pores 7) is longer than the lesion. To ensure that the balloon is sufficiently long to cover the lesion and extend beyond the region, a surgeon performing the method may first determine the length of the lesion, and choose and insert a balloon with a porous region of sufficient length to extends along the entirety of the lesion and also extends both distally and proximally of the lesion, and operate the system to force the suspension of nanoparticles into the into the balloon and through the pores to the blood vessel wall along the entirety of the lesion and portions of the blood vessel wall extending both distally and proximally of the lesion. Alternately, several balloons shorter than the lesion, or several applications of a single balloon shorter that the lesion, may be used in the method.

While the preferred embodiments of the devices and methods have been described in reference to the environment in which they were developed, they are merely illustrative of the principles of the inventions. The elements of the various embodiments may be incorporated into each of the other species to obtain the benefits of those elements in combination with such other species, and the various beneficial features may be employed in embodiments alone or in combination with each other. Other embodiments and configurations may be devised without departing from the spirit of the inventions and the scope of the appended claims.

1. A balloon catheter system comprising:
a catheter comprising a catheter shaft having a distal end and a proximal end, with a balloon disposed on the distal end, said balloon having a balloon wall with a plurality of pores communicating through the balloon wall;
a reservoir containing a suspension of nanoparticles in solution;
an inflator, operable to force the suspension of nanoparticles through the catheter and through the balloon wall;
wherein the pores have a conical cross section, along a long axis of the pores through the balloon wall.

2. The balloon catheter system of claim 1 wherein:
the pores have a first diameter at an inside surface of the balloon in the range of 3 to 8 μm, and a second diameter at an outside surface of the balloon in the range of 7 to 16 μm.

3. The balloon catheter system of claim 1 wherein:
the pores have a first diameter at an inside surface of the balloon in the range of 3 to 8 μm, and a second diameter at an outside surface of the balloon in the range of 7 to 16 μm; and
the nanoparticles in the suspension have a diameter in the range of 40 to 250 nm.

4. The balloon catheter system of claim 1 wherein:
the pores have a first diameter at an inside surface of the balloon in the range of 3 to 8 μm, and a second diameter at an outside surface of the balloon in the range of 7 to 16 μm; and
the nanoparticles in the suspension have a diameter in the range of 60 to 120 nm.

5. The balloon catheter system of claim 3 wherein:
the nanoparticles comprise micelles loaded with a therapeutic agent.

6. The balloon catheter system of claim 3 wherein:
the ratio of micelle diameter to the first diameter is in the range of about 0.08 to 1 (1 to 12) to about 0.005 to 1 (1 to 200).

7. The balloon catheter system of claim 3 or 4 wherein:
the ratio of micelle diameter to the first diameter is about 1 to 20.

8. The balloon catheter system of claim 3 or 4 wherein:
the inflator is operable to apply 6 to 16 atmospheres of pressure to the reservoir.

9. The balloon catheter system of claim 3 or 4 wherein:
the inflator is operable to apply 6 to 12 atmospheres of pressure to the reservoir.

10. The balloon catheter system of claim 5 wherein:
the inflator is operable to apply 6 to 8 atmospheres of pressure for a first period, and subsequently apply 12-14 atmospheres of pressure for a second period.

11. The balloon catheter system of claim 3 or 4 wherein:
the ratio of average diameter of a nanoparticles to the total pore area on the inside surface of the balloon wall is in the range of 0.0000016 to 1 to 0.0008 to 1.

12. A balloon catheter system comprising:
a catheter comprising a catheter shaft having a distal end and a proximal end, with a balloon disposed on the distal end, said balloon having a balloon wall with a plurality of pores communicating through the balloon wall;
a reservoir containing a suspension of nanoparticles in solution;
an inflator, operable to force the suspension of nanoparticles through the catheter and through the balloon wall;
wherein a ratio of an average size of the nanoparticles to a total pore area on an inside surface of the balloon wall is in the range of 0.0000016 to 1 to 0.0008 to 1.

13. The balloon catheter system of claim 12, wherein:
average size of the nanoparticles is in the range of 40 to 250 nm; and
the total pore area on an inside surface of the balloon wall is in the range of 900 to 30,000 microns.

14. The balloon catheter system of claim 11, wherein:
average size of the nanoparticles is in the range of 40 to 250 nm; and
the pores have an average size of 3 to 8 μm on an inside wall of the balloon; and
the number of pores is in the range of 100 to 1000.

15. The balloon catheter system of claim 12, wherein:
the nanoparticles comprise micelles loaded with a therapeutic agent.

16. The balloon catheter system of any of claims 12 through 15, wherein:
the nanoparticles are loaded with a therapeutic agent, and said therapeutic agent comprises at least one of rapamycin or rapamycin analogs, ABT-578, zotarolimus, everolimus, biolimus A9, deforolimus, temsirolimus, tacrolimus, pimcrolimus, nitric oxide synthase, C3 exoenzyme, RhoA inhibitors, tubulusin, A3 agonists, CB2 agonists, 17-AAG, Hsp90 antagonists, tyrphostins, cathepsin S inhibitors, paclitaxel, corticosteroids, glucocorticoids, dexamethasone, ceramides, dimethyl sphingosine, ether-linked diglycerides, ether-linked phosphatidic acids, sphinganines, estrogens, taxol, taxol analogs, actinomycin D, prostaglandins, vitamin A, probucol, or Batim.

17. The balloon catheter system of claim 5 wherein:
the therapeutic agent comprises at least one of rapamycin or rapamycin analogs, ABT-578, zotarolimus, everolimus, biolimus A9, deforolimus, temsirolimus, tacrolimus, pimcrolimus, nitric oxide synthase, C3 exoenzyme, RhoA inhibitors, tubulusin, A3 agonists, CB2 agonists, 17-AAG, Hsp90 antagonists, tyrphostins, cathepsin S inhibitors, paclitaxel, corticosteroids, glucocorticoids, dexamethasone, ceramides, dimethyl sphingosine, ether-linked diglycerides, ether-linked phosphatidic acids, sphinganines, estrogens, taxol, taxol analogs, actinomycin D, prostaglandins, vitamin A, probucol, or Batim.

18. A method of treating a diseased blood vessel in a patient, said method comprising:
inserting a balloon of a balloon catheter system into the blood vessel, where said balloon comprises a balloon wall with a plurality of pores communicating through the balloon wall, and said pores have a conical cross section, along an axis of the pores passing from an inside surface of the balloon wall to an outside surface of the balloon wall;
forcing a suspension of nanoparticles into the balloon and through the pores to a blood vessel wall.

19. The method of claim 18, wherein:
the pores have a first diameter at an inside surface of the balloon in the range of 3 to 8 μm, and a second diameter at an outside surface of the balloon in the range of 7 to 16 μm.

20. The method of claim 19, wherein:
the nanoparticles in the suspension have a diameter in the range of 40 to 250 nm.

21. The method of claim 19, wherein:
the nanoparticles in the suspension have a diameter in the range of 60 to 120 nm.

22. The method of claim 20 or 21, wherein:
the nanoparticles comprise micelles loaded with a therapeutic agent.

23. The method of claim 22, wherein:
the step of forcing the suspension of nanoparticles into the balloon comprises forcing the suspension of nanoparticles into the balloon at a pressure in the range of 6 to 16 atmospheres of pressure, more preferably 6 to 12 atmospheres of pressure.

24. The method of claim 22, wherein:
the step of forcing the suspension of nanoparticles into the balloon comprises forcing the suspension of nanoparticles into the balloon at a pressure in the range of 6 to 8 atmospheres of pressure for a first period, and subsequently at a pressure in the range of 12 to 14 atmospheres for a second time period.

25. The method of claim 18, wherein:
the step of forcing a suspension of nanoparticles into the balloon and through the pores to a blood vessel wall is accomplished to provide a flow rates of 0.0005 to 0.038 ml/sec of the suspension out of the balloon.

26. The method of claim 18, wherein:
the step of forcing a suspension of nanoparticles into the balloon and through the pores to a blood vessel wall is accomplished to provide a flow rates of 0.0033 to 0.0375 ml/sec of the suspension out of the balloon.

27. The method of claim 22, wherein:
the therapeutic agent comprises at least one of rapamycin or rapamycin analogs, ABT-578, zotarolimus, everolimus, biolimus A9, deforolimus, temsirolimus, tacrolimus, pimcrolimus, nitric oxide synthase, C3 exoenzyme, RhoA inhibitors, tubulusin, A3 agonists, CB2 agonists, 17-AAG, Hsp90 antagonists, tyrphostins, cathepsin S inhibitors, paclitaxel, corticosteroids, glucocorticoids, dexamethasone, ceramides, dimethyl sphingosine, ether-linked diglycerides, ether-linked phosphatidic acids, sphinganines, estrogens, taxol, taxol analogs, actinomycin D, prostaglandins, vitamin A, probucol, or Batim.

28. The method of claim 18, further comprising the steps of:
determining a length of a lesion in the blood vessel to be treated;
performing the step of inserting the balloon of the balloon catheter wherein the balloon has a porous region of a length sufficient such that, when disposed within the vessel to be treated and proximate the lesion, the porous region extends along the entirety of the lesion and also extends both distally and proximally of the lesion.

29. The balloon catheter system of claim 4 wherein:
the nanoparticles comprise micelles loaded with a therapeutic agent.

30. The balloon catheter system of claim 4 wherein:
the ratio of micelle diameter to the first diameter is in the range of about 0.08 to 1 (1 to 12) to about 0.005 to 1 (1 to 200).
